(19) United States
(12) Patent Application Publication
(10) Pub. No.: US 2009/0054738 A1
(43) Pub. Date: Feb. 26, 2009

(54) SYSTEM AND METHOD FOR PHYSIOLOGICAL ANALYSIS AND FEEDBACK APPLICATION THEREOF

(75) Inventors: Tze-Chin CHOU, Hsinchu City (TW); Chun-I Chu, Hsinchu County (TW)

Correspondence Address: Muncy, Geissler, Olds & Lowe, PLLC, P.O. BOX 1364, FAIRFAX, VA, 22038-1364, US

(73) Assignee: Industrial Technology Research Institute

(21) Appl. No.: 12/163,195

(22) Filed: Jun. 27, 2008

(30) Foreign Application Priority Data
Aug. 22, 2007 (TW) 96131043

(51) Int. Cl. A61B 5/00; G06Q 50/00

(52) U.S. Cl. 600/300; 705/3

(57) ABSTRACT

A system for analyzing physiological information and providing feedback is provided. An interface receives time information recording a time point, physiological information of a first user measured at the time point, and a purpose setting determined by the first user. A processor determines a physiological status according to the time information, the physiological information and a physiological information history, selects one of service providers having service attributes conforming to purpose setting and the physiological status according to the purpose setting and the physiological status, and sends service information corresponding to the selected service provider to the first user.

This Application claims priority of Taiwan Patent Application No. 96131043, filed on 22 Aug. 2007, the entirety of which is incorporated by reference herein.

### BACKGROUND

The invention relates to monitoring physiological status and providing commercial services, and in particular to a system and method for providing services according to a user's birth control setting and physiological status.

Basal body temperature is utilized in birth control. The basal body temperature is the body temperature measured immediately after awakening and before any physical activity has been undertaken. Monitoring of basal body temperature is one way of estimating the day of ovulation. Accordingly, charting of basal body temperature may be used in some methods of fertility awareness, and may be used to determine the onset of post-ovulatory infertility.

It requires much attention and care to keep a record of the basal temperature.

Accordingly, a system and method providing a convenient way of monitoring basal body temperature, as well as providing services pertaining to birth control is needed.

### SUMMARY

A system for (analyzing) physiological information and providing feedback is provided, comprising an interface, a storage device, and a processor. The interface receives time information recording a time point, physiological information of a first user measured at the time point, and a purpose setting determined by the first user. The storage device comprises a user information storage unit and a service provider information storage unit. The user information storage unit stores the time information, the physiological information, the purpose setting, and a physiological information history corresponding to the first user, wherein the physiological information history comprises a plurality of items of time information and corresponding physiological information which preceded the time point. The service provider information storage unit stores service information and service attributes corresponding to a plurality of service providers. The processor determines a physiological status according to the time information, the physiological information and the physiological information history, selects one of the service providers having service attributes conforming to the purpose setting and the physiological status according to the purpose setting and the physiological status, and sends the service information corresponding to the selected service provider to the first user.

A method for analyzing physiological information and providing feedback is also provided. Time information recording a time point, physiological information of a first user measured at the time point, and a purpose setting determined by the first user are received. The time information, the physiological information, the purpose setting, and a physiological information history corresponding to the first user are stored, wherein the physiological information history comprises a plurality of items of time information and corresponding physiological information which preceded the time point. Service information and service attributes corresponding to a plurality of service providers are stored. A physiological status is determined according to the time information, the physiological information and the physiological information history. One of the service providers having service attributes conforming to the purpose setting and the physiological status is selected according to the purpose setting and the physiological status. The service information corresponding

### BRIEF DESCRIPTION OF DRAWINGS

FIG. 1 illustrates a schematic view of an embodiment of a system for analyzing physiological information and providing feedback;

FIGS. 2A and 2B illustrate a flowchart of an embodiment of a method for analyzing physiological information and providing feedback; and

FIG. 3 is a flowchart of an embodiment of a method of estimating ovulation status.

### DETAILED DESCRIPTION

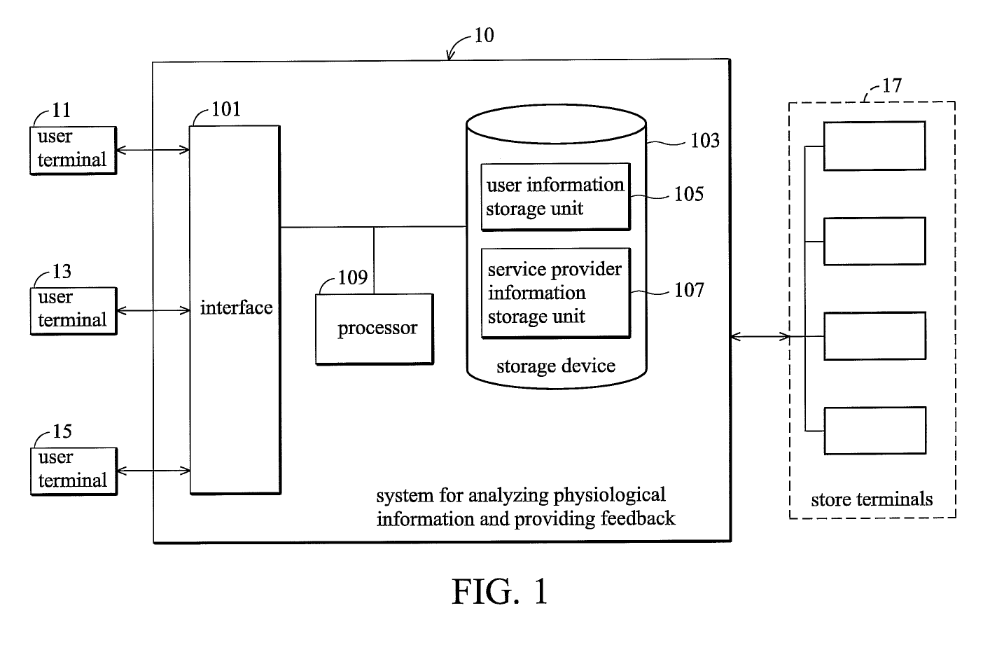

FIG. 1 illustrates a schematic view of an embodiment of a system for analyzing physiological information and providing feedback. The system for analyzing physiological information and providing feedback 10 (hereinafter referred to as system 10) is a system providing services to a user according to a birth control setting and physiological status of the user. The system 10 connects to user terminals 11, 13, 15 via a wired/wireless network, and connects to a plurality of store terminals 17.

The user terminal 11 can be a wrist watch or mobile phone performing functions of a clock and a thermometer. The user terminal 11 measures a basal body temperature of a first user wearing the user terminal 11 at a particular time (for example, a particular time in the morning). In addition, the user terminal 11 uploads, automatically or in response to a user command, the measured basal body temperature (hereinafter referred to as temperature information) and the time information recording a time point at which the temperature is measured to the system 10.

The user terminal 13 can be a computer. The first user can set identification information and a birth control setting in the system 10 through the user terminal 13.

The system 10 comprises: an interface 101, a storage device 103, and a processor 109.

The interface 101 receives the time information, temperature information of the first user measured at the time point, and the birth control setting determined by the first user.

The storage device 103 comprises a user information storage unit 105 and a service provider information storage unit 107.

The user information storage unit 105 stores the time information, the temperature information, the birth control setting, and a temperature information history corresponding to the first user. The temperature information history comprises a plurality of items of time information and corresponding temperature information which preceded the time point, i.e., a record of the basal body temperature of the first user measured during past days or months.

In addition, the user information storage unit 105 further stores first identification information and/or first contact information of the first user, as well as a data publication setting of the first user, i.e., if the first user is willing to make the first contact information public to selected service provider.

If the first user is willing to share the information such as the estimated ovulation status with another user (referred to as second user), the user information storage unit 105 further stores authorization information, comprising a second identification information and/or second contact information corresponding to a second user. The authorization information further comprises an authorization password and an information sharing setting for the second user, specifying which part of the information pertaining to the first user is available for the second user.

The service provider information storage unit 107 stores service information and service attributes corresponding to a plurality of service providers (stores). For example, the service provider information storage unit 107 stores contact information (such as an address, a telephone number, and a website address) of service providers, such as an obstetrics and gynecology clinic, an institute providing postpartum services, a baby commodities shop, a drug store, a pharmacy, a flower shop, a restaurant, a motel, a book store, etc. In addition, at least one service attribute is set for each service provider, such as a user birth control setting and a user ovulation status setting. The user ovulation status setting can be set as one of the following: infertile phase (S), fertile phase (D), pregnancy (P), and abnormal (U). The user birth control setting can be set as trying to conceive (Y) or avoiding pregnancy (N) Accordingly, the service attribute of the service provider can comprise at least one of the following combinations: YS, YD, YP, YU, NS, ND, NP, and NU. The service attribute of the service provider can comprise a plurality of combinations. For example, an obstetrics and gynecology clinic can have service attributes of both YP and NP. Accordingly, under circumstances of YP or NP, the service information of the obstetrics and gynecology clinic is provided to the first user.

The service attributes stored in the service provider information storage unit 107 can comprise an electronic coupon.

The processor 109 determines an estimated ovulation status according to the time information, the temperature information and the temperature information history stored in the user information storage unit 105, selects one of the service providers having service attributes conforming to the birth control setting and the estimated ovulation status according to the birth control setting and the estimated ovulation status, and sends the service information corresponding to the selected service provider to the first user via an electronic mail, a message, etc.

For example, if the first user sets the birth control setting as “trying to conceive (Y)”, and the estimated ovulation status for the current day is “pregnancy (P)”, the service provider having a service attribute of YP is selected. The service information corresponding to the selected service provider is sent to the first user via an electronic mail, a message, etc.

If the data publication setting of the first user is set as “willing to make the first contact information public to selected service provider”, the processor 109 further transmits the first identification information and/or the first contact information of the first user to the selected service provider.

If the first user is willing to share information with the second user, the processor 109 provides the service information of the selected service provider to the second user according to the authorization information. In addition, when the processor 109 receives the second identification information and the authorization password, at least one of the temperature information history, the time information, and the temperature information is displayed accordingly.

For example, the user terminal 15 is a computer. When the second identification information and the authorization password is input into the user terminal 15 by the second user, at least one of the temperature information history, the time information, and the temperature information is displayed is displayed on a screen of the user terminal 15 according to the authorization information.

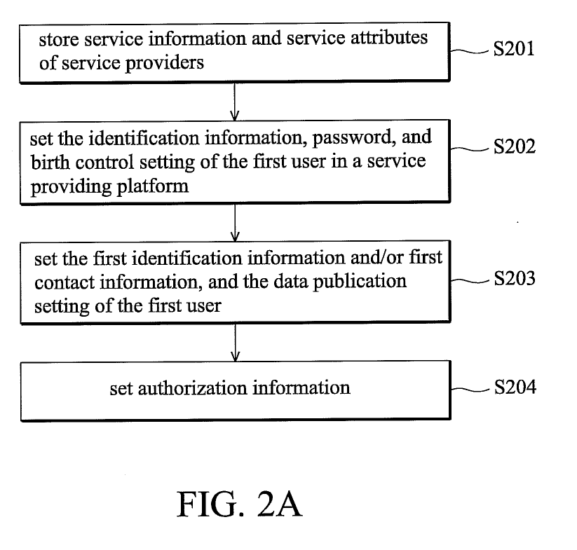

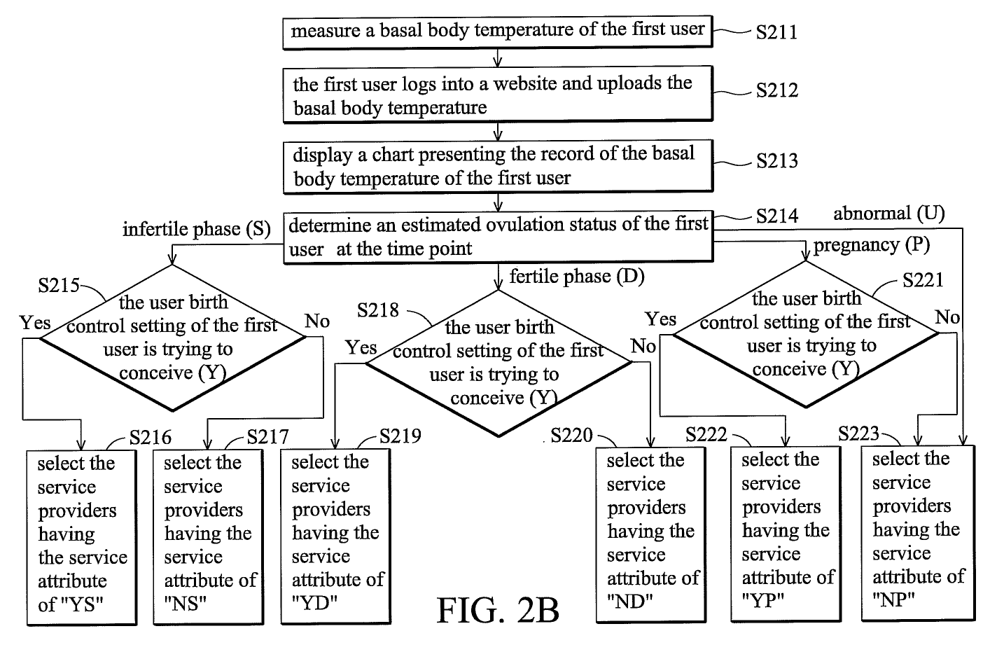

FIGS. 2A and 2B illustrate a flowchart of an embodiment of a method for analyzing physiological information and providing feedback.

Referring to FIG. 2A, in step S201, service information and service attributes of a plurality of service providers are stored. For example, contact information (such as an address, a telephone number, and a website address) of service providers is stored, such as an obstetrics and gynecology clinic, an institute providing postpartum services, a baby commodities shop, a drug store, a pharmacy, a flower shop, a restaurant, a motel, a book store, etc. In addition, at least one service attribute is set for each service provider, such as a user birth control setting and a user ovulation status setting. The user ovulation status setting can be set as one of the following: infertile phase (S), fertile phase (D), pregnancy (P), and abnormal (U). The user birth control setting can be set as trying to conceive (Y) or avoiding pregnancy (N). Accordingly, the service attribute of the service provider can comprise at least one of the following combinations: YS, YD, YP, YU, NS, ND, NP, and NU. The service attribute of the service provider can comprise a plurality of combinations. For example, an obstetrics and gynecology clinic can have service attributes of both YP and NP. Accordingly, under circumstances of YP or NP, the service information of the obstetric and gynecologic clinic is provided to the first user. In addition, the service attribute comprises an electronic coupon.

In step S202, the identification information, password, and birth control setting of the first user is set in a service providing platform.

In step S203, the first identification information and/or first contact information, and the data publication setting of the first user is set. The data publication setting specifies whether the first user is willing to make the first contact information public to selected service provider.

In step S204, if the first user is willing to share the information, such as the estimated ovulation status with another user (referred to as second user), authorization information corresponding to the second user can be set. The authorization information comprises the second identification information and/or second contact information, an authorization password, and an information sharing setting corresponding to a second user. The information sharing setting specifies which part of the information pertaining to the first user is available for the second user.

Referring to FIG. 2B, in step S211, a basal body temperature of the first user is measured. The measurement is automatically performed by a wrist watch or mobile phone performing functions of a clock and a thermometer at a particular time point (for example, a particular time in the morning).

In step S212, the first user logs into a website and uploads the basal body temperature. Here, a record of the basal body temperature of the first user has been kept for a period of time, i.e., a temperature information history corresponding to the first user exists. The temperature information history comprises a plurality of items of time information and corresponding temperature information which preceded the time point, i.e., a record of the basal body temperature of the first user measured during past days or months.

In step S213, a chart presenting the record of the basal body temperature of the first user is displayed. Here, the chart presents the temperature information history and the basal body temperatures measured at different time points.

In addition, if the first user is willing to share the information such as the estimated ovulation status with another user (referred to as second user), and the corresponding authorization information has been set, the temperature information history and/or other information is displayed in response to receiving the second identification information and the authorization password (not shown in FIG. 2B).

In step S214, the temperature information history and the temperature information of the current day is analyzed, and an estimated ovulation status of the first user at the time point (for example, the current day) is accordingly determined. The estimated ovulation status is one of the following: infertile phase (S), fertile phase (D), pregnancy (P), and abnormal (U).

The method proceeds to step S215 when the estimated ovulation status is infertile phase (S); the method proceeds to step S218 when the estimated ovulation status is fertile phase (D); the method proceeds to step S221 when the estimated ovulation status is pregnancy (P); the method proceeds to step S223 when the estimated ovulation status is abnormal (U).

In step S215, it is determined whether the user birth control setting of the first user is trying to conceive (Y), and if so, the method proceeds to step S216, otherwise, the method proceeds to step S217. In step S216, the service providers having the service attribute of “YS” (as shown in FIG. 2A) are selected, and the service information of the selected service providers are sent to the first user and/or the second user. If none of the service providers has the service attribute “YS”, no further information is presented to the first user and/or the second user. In step S217, the service providers having the service attribute of “NS” (as shown in FIG. 2A) are selected, and the service information of the selected service providers are sent to the first user and/or the second user. For example, the service providers having the service attribute “NS” may comprise a restaurant, a hotel, and a flower shop.

In step S218, it is determined whether the user birth control setting of the first user is trying to conceive (Y), and if so, the method proceeds to step S219, otherwise, the method proceeds to step S220. In step S219, the service providers having the service attribute of “YD” (as shown in FIG. 2A) are selected, and the service information of the selected service providers are sent to the first user and/or the second user. For example, the service providers having the service attribute “YD” may comprise a restaurant, a hotel, and a flower shop. In step S220, the service providers having the service attribute of “ND” (as shown in FIG. 2A) are selected, and the service information of the selected service providers are sent to the first user and/or the second user. For example, the service providers having the service attribute “ND” may comprise a pharmacy.

In step S221 it is determined whether the user birth control setting of the first user is trying to conceive (Y), and if so, the method proceeds to step S222, otherwise, the method proceeds to step S223. In step S222, the service providers having the service attribute of “YP” (as shown in FIG. 2A) are selected, and the service information of the selected service providers are sent to the first user and/or the second user. For example, the service providers having the service attribute “YP” may comprise an obstetrics and gynecology clinic and/or an institute providing postpartum services. In step S223, the service providers having the service attribute of “NP” (as shown in FIG. 2A) are selected, and the service information of the selected service providers are sent to the first user and/or the second user. For example, the service providers having the service attribute “NS” may comprise a hospital and/or an obstetrics and gynecology clinic.

In step S214, if the estimated ovulation status is abnormal (U), the method proceeds to step S223 without further determining the birth control setting of the first user. In step S223, the service providers having the service attribute of “YU” and “NU” (as shown in FIG. 2A) are selected, and the service information of the selected service providers are sent to the first user and/or the second user. For example, the service providers having the service attribute “YU” and “NU” may comprise an obstetrics and gynecology clinic.

In addition, if the data publication setting of the first user is set as “willing to make the first contact information public to selected service provider”, the first identification information and/or the first contact information of the first user and/or the second user are transmitted to the service providers selected in steps S216, S217, S219, S220, S222, and S223 according to the data publication setting.

The method for determining the estimated ovulation status of the first user at the time point (for example, the current day) can be implemented based on a record of the basal body temperatures. A menstrual cycle can be divided into 3 phases according to the basal body temperature, i.e., infertile phase (post-ovulatory infertility), fertile phase (ovulation, within 2˜3 days from a rise in basal body temperature), and a relatively infertile phase (pre-ovulatory infertility). Generally, it is not easy to conceive during the infertile phase; and it is easy to conceive during the fertile phase. Normal sperm life is up to 3˜4 days, making prediction of ovulation several days in advance necessary for avoiding pregnancy. According to the described method, while avoiding pregnancy, the period during which to abstain from sexual intercourse comprises the relatively infertile phase and fertile phase.

In addition, 16 or more consecutive days of elevated temperatures mean that a woman is almost certainly pregnant.

In addition, when no rise in basal body temperature occurs in 28 or more consecutive days, the estimated ovulation status is determined as “abnormal”, and information pertaining to an obstetrics and gynecology clinic is sent to the corresponding user.

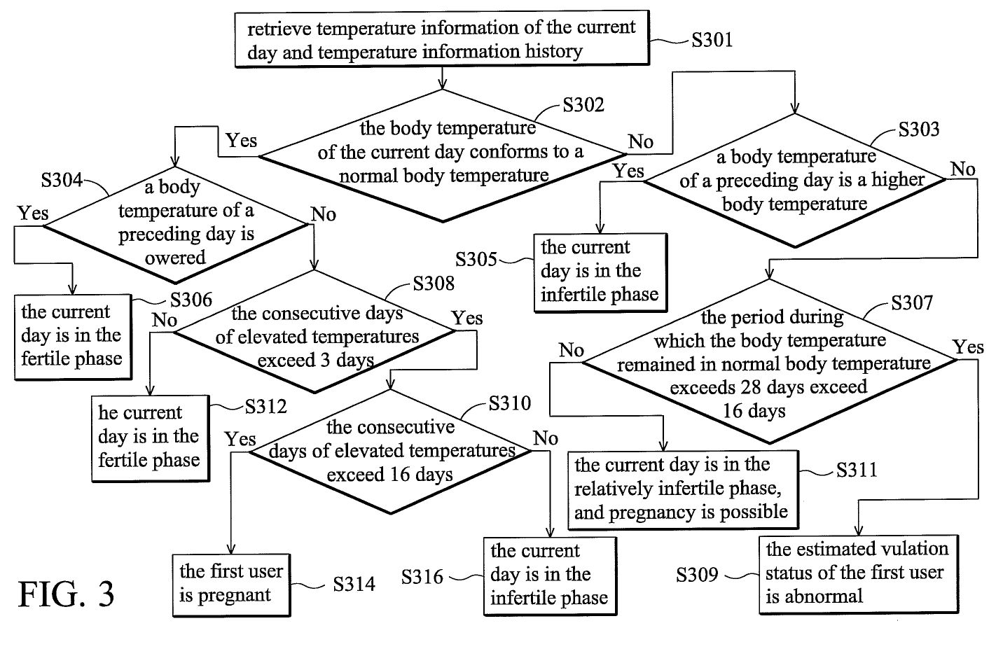

FIG. 3 is a flowchart of an embodiment of a method of estimating ovulation status of the first user performed by the processor 109.

In step S301, temperature information of the current day and temperature information history are retrieved. In step s302, it is determined whether the body temperature of the current day conforms to a normal body temperature, and if so, the method proceeds to step S303, otherwise, the method proceeds to step S304.

In step S303, it is determined whether a body temperature of a preceding day is a higher body temperature, and if so, the method proceeds to step S305, otherwise, the method proceeds to step S307. In step S305, it is determined whether the current day is in the infertile phase, i.e., the estimated ovulation status is infertile phase (S). In step S307, it is determined whether the period during which the body temperature remained in normal body temperature exceeds 28 days, and if so, the method proceeds to step S309, otherwise, the method proceeds to step S311. In step S309, it is determined that the estimated ovulation status of the first user is abnormal. In step S311, it is determined that the current day is in the relatively infertile phase, and pregnancy is possible. In this circumstance, the estimated ovulation status is infertile phase (S) or pregnancy (P), and further confirmation is required.

In step S304, it is determined whether a body temperature of a preceding day is lowered, and if so, the method proceeds to step S306, otherwise, the method proceeds to step S308. In step S306, it is determined that the current day is in the fertile phase, i.e., the estimated ovulation status is fertile phase (D). In step S308, it is determined whether the consecutive days of elevated temperatures exceed 3 days, and if so, the method proceeds to S310, otherwise, the method proceeds to S312. In step S312, it is determined the current day is in the fertile phase, i.e., the estimated ovulation status is fertile phase (D). In step S310, it is determined whether the consecutive days of elevated temperatures exceed 16 days, and if so, the method proceeds to 314, otherwise, the method proceeds to S316. In step S314, it is determined the first user is pregnant, i.e., the estimated ovulation status is pregnancy (P). In step S316, it is determined the current day is in the infertile phase, i.e., the estimated ovulation status is infertile phase (S).

According to the described embodiment, estimating ovulation status is used as an example. The system method for analyzing physiological information and providing feedback of the invention can be implemented for estimating other physiological statuses using other physiological information, and providing corresponding information and services.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A system for analyzing physiological information and providing feedback, comprising:
an interface receiving time information recording a time point, physiological information of a first user measured at the time point, and a purpose setting determined by a first user;
a storage device comprising:
a user information storage unit storing the time information, the physiological information, the purpose setting, and a physiological information history corresponding to the first user, wherein the physiological information history comprises a plurality of items of time information and corresponding physiological information which preceded the time point; and
a service provider information storage unit storing service information and service attributes corresponding to a plurality of service providers; and
a processor determining a physiological status according to the time information, the physiological information and the physiological information history, selecting one of the service providers having service attributes conforming to the purpose setting and the physiological status according to the purpose setting and the physiological status, and sending the service information corresponding to the selected service provider to the first user.

2. The system for analyzing physiological information and providing feedback of claim 1, wherein the physiological information comprises body temperature information.

3. The system for analyzing physiological information and providing feedback of claim 1, wherein the purpose setting comprises a birth control setting.

4. The system for analyzing physiological information and providing feedback of claim 1, wherein the service attributes stored in the service provider information storage unit comprises a user purpose setting and a user ovulation status setting, wherein the processor further selects the service provider having the user purpose setting and the user ovulation status setting conforming to the purpose setting and the physiological status of the first user, and the processor further provides the service information of the selected service provider to the first user.

5. The system for analyzing physiological information and providing feedback of claim 1, wherein the service attributes stored in the service provider information storage unit comprises an electronic coupon.

6. The system for analyzing physiological information and providing feedback of claim 1, wherein the user information storage unit further stores first identification information and/or first contact information of the first user, wherein the processor further transmits the first identification information and/or the first contact information of the first user to the selected service provider.

7. The system for analyzing physiological information and providing feedback of claim 1, wherein the user information storage unit further stores first identification information and a first password of the first user, wherein the interface displays at least one of the physiological information history, the time information, and the physiological information in response to receiving the first identification information and the first password.

8. The system for analyzing physiological information and providing feedback of claim 1, wherein the user information storage unit further stores authorization information, comprising a second identification information and/or second contact information corresponding to a second user, and wherein the processor further provides the service information of the selected service provider to the second user according to the authorization information.

9. The system for analyzing physiological information and providing feedback of claim 8, wherein the authorization information comprises an authorization password and a information sharing setting for a second user, specifying which part of the information pertaining to the first user is available for the second user, wherein the interface displays at least one of the physiological information history, the time information, and the physiological information in response to receiving the second identification information and the authorization password.

10. The system for analyzing physiological information and providing feedback of claim 1, wherein the physiological status comprises an estimated ovulation status.

11. The system for analyzing physiological information and providing feedback of claim 10, wherein the estimated ovulation status specifies one of the following: infertile phase, fertile phase, pregnancy, and abnormal

12. A method for analyzing physiological information and providing feedback, comprising:
receiving time information recording a time point, physiological information of a first user measured at the time point, and a purpose setting determined by the first user;
storing the time information, the physiological information, the purpose setting, and a physiological information history corresponding to the first user, wherein the physiological information history comprises a plurality of items of time information and corresponding physiological information which preceded the time point;
storing service information and service attributes corresponding to a plurality of service providers;
determining a physiological status according to the time information, the physiological information and the physiological information history;
selecting one of the service providers having service attributes conforming to the purpose setting and the physiological status according to the purpose setting and the physiological status; and
presenting the service information corresponding to the selected service provider to the first user.

13. The method for analyzing physiological information and providing feedback of claim 12, wherein the physiological information comprises body temperature information.

14. The method for analyzing physiological information and providing feedback of claim 12, wherein the purpose setting comprises a birth control setting.

15. The method for analyzing physiological information and providing feedback of claim 12, wherein the service attributes comprises a user purpose setting and a user ovulation status setting, wherein the method further selects the service provider having the user purpose setting and the user ovulation status setting conforming to the purpose setting and the physiological status of the first user, and provides the service information of the selected service provider to the first user.

16. The method for analyzing physiological information and providing feedback of claim 12, wherein the service attributes comprises an electronic coupon.

17. The method for analyzing physiological information and providing feedback of claim 12, further storing first identification information and/or first contact information of the first user, wherein the method further transmits the first identification information and/or the first contact information of the first user to the selected service provider.

18. The method for analyzing physiological information and providing feedback of claim 12, further storing authorization information comprising a second identification information and/or second contact information corresponding to a second user, and providing the service information of the selected service provider to the second user according to the authorization information.

19. The method for analyzing physiological information and providing feedback of claim 18, wherein the authorization information comprises an authorization password and a information sharing setting for a second user, specifying which part of the information pertaining to the first user is available for the second user, wherein the interface displays at least one of the physiological information history, the time information, and the physiological information in response to receiving the second identification information and the authorization password.

20. The method for analyzing physiological information and providing feedback of claim 12, further storing first identification information and a first password of the first user, and displaying at least one of the physiological information history, the time information, and the physiological information in response to receiving the first identification information and the first password.

21. The method for analyzing physiological information and providing feedback of claim 12, wherein the physiological status comprises an estimated ovulation status.

22. The method for analyzing physiological information and providing feedback of claim 12, wherein the estimated ovulation status specifies one of the following: infertile phase, fertile phase, pregnancy, and abnormal.
